(19) United States
(12) Patent Application Publication
(10) Pub. No.: US 2023/0248378 A1
(43) Pub. Date: Aug. 10, 2023

(54) CATHETER BASED DEVICE FOR TREATMENT OF OBSTRUCTION IN BODY LUMEN

(71) Applicant: Althea Medical Ltd., Ariel (IL)

(72) Inventors: Raphael Benary, Tel Aviv (IL); Lia Ofek, Ramat Hasharon (IL); Or Chernin, Rosh Ha'Ayin (IL)

(73) Assignee: Althea Medical Ltd., Ariel (IL)

(21) Appl. No.: 18/004,704

(22) PCT Filed: Jul. 22, 2021
(86) PCT No.: PCT/US2021/042676

§ 371 Date: Jan. 9, 2023

Related U.S. Application Data
(60) Provisional application No. 63/202,183, filed on May 30, 2021.
(60) Provisional application No. 63/055,357, filed on Jul. 23, 2020.

(51) Int. Cl. A61B 17/221

(52) U.S. Cl. CPC A61B 17/221; A61B 2017/22084

(57) ABSTRACT

A method and apparatus for treating or removing an obstruction in the body lumen of a patient, and particularly the treatment of a pulmonary embolism is disclosed. The treatment involves macerating and dissolving the clot by providing mechanical means of breaking up the obstruction while applying a dissolving agent such as a lytic agent to the specific area. A means of aspirating the obstruction debris is provided. In addition, various means of grasping an obstruction and removing it from the body lumen are disclosed.

### FIELD OF THE INVENTION

The present invention generally relates to devices and methods for treatment of obstructions in body lumens.

### BACKGROUND

Thromboembolism is the formation in a blood vessel of a clot (thrombus) that breaks loose (embolizes) and is carried by the blood stream to another location in the circulatory system resulting in a clot or obstruction at that new location. For example, a clot may embolize and plug a vessel in the lungs, a condition called pulmonary embolism. Thromboembolism is a significant cause of morbidity and mortality, especially in adults. A thromboembolism can be sudden and massive and can happen at any time.

When blood clots form in the venous circulation of the body they may move or embolize to the lungs. The clots typically embolize from the veins of the legs, pelvis, or inferior vena cava to the right heart cavities and thence into the pulmonary arteries. This results in right heart failure and decreased blood flow through the lungs with subsequent decreased oxygenation of the lungs, heart and the rest of the body. When clots enter the pulmonary arteries, obstruction and spasm of the different arteries of the lung occur, which further decrease blood flow and gaseous exchange through the lung tissue resulting in pulmonary edema.

### SUMMARY OF THE INVENTION

The present invention seeks to provide devices and methods for treatment of obstructions in body lumens.

There is provided in accordance with an embodiment of the invention a device that includes an inner tube linearly and rotationally movable in an outer shaft, the inner tube being formed with nozzle holes and slots, disruption elements extendable outwards through the slots, and an aspiration device coupled to a proximal portion of the outer shaft.

Further embodiments are described below.

### BRIEF DESCRIPTION OF THE DRAWINGS

The present invention will be understood and appreciated more fully from the following detailed description, taken in conjunction with the drawings in which:

FIG. 1 is a simplified illustration of a catheter-based treatment device (CBTD) in accordance with a non-limiting embodiment of the invention.

FIG. 2 is a simplified illustration of an inner tube being advanced from within an outer shaft of the device.

FIG. 3 is a simplified illustration of the inner tube having slots and nozzle holes.

FIG. 4 is a simplified illustration of the inner tube being advanced into an obstruction so that the slots and nozzle holes are located within the obstruction.

FIG. 5 is a simplified illustration of the inner tube within the obstruction.

FIG. 6 is a simplified illustration of the CBTD with a centering element designed to ensure that the inner tube is inserted into the obstruction rather than between the obstruction and the body lumen (not shown).

FIG. 7 is a simplified illustration of another possible feature of the present embodiment in which the outer shaft has a balloon that can expand and form a seal against the inner diameter of the body lumen.

FIG. 8 is a simplified illustration of a catheter-based treatment device (CBTD) in accordance with another non-limiting embodiment of the invention.

FIG. 9 is a simplified illustration of another view of the CBTD of FIG. 8.

FIG. 10 is a simplified illustration of an inner element extending from within the outer shaft of the CBTD of FIG. 8.

FIG. 11 is a simplified illustration of a distal capture mesh.

FIG. 12 is a simplified illustration of the distal end of the capture mesh.

FIG. 13 is a simplified enlarged illustration of the capture mesh as it folds over the obstruction.

FIG. 14 is a simplified illustration of a catheter-based treatment device (CBTD) in accordance with another non-limiting embodiment of the invention.

FIG. 15 is a simplified illustration of the obstruction grasped in the grasping arms of the device.

FIG. 16 is a simplified illustration of a grasping element having operating arms which are cut from the struts of the grasping arms.

FIG. 17 is a simplified illustration of an aspiration system, in accordance with a non-limiting embodiment of the invention, including an aspiration hub, an aspiration port and an aspiration tube.

FIG. 18 is a simplified illustration of an expander element being inserted into the aspiration system through the aspiration hub.

FIG. 19 is a simplified illustration of an expander element of the aspiration system.

FIG. 20 is a simplified illustration of another expander element used in the aspiration system.

FIG. 21 is a simplified illustration of the aspiration system with both distal and proximal sections having approximately the same diameter following the expanding process.

FIG. 22 is a simplified illustration of a fully expanded aspiration system proximal to a thrombus and prior to aspiration.

FIG. 23 is a simplified illustration of a cross section of a distal section of a portion of the aspiration system.

FIG. 24 is a simplified illustration of another method of achieving the expansion of the distal section.

FIG. 25 is a simplified illustration of the relations between the lengths of the distal section and the proximal section.

FIG. 26 is a simplified illustration of another embodiment of an expanding aspiration system.

FIG. 27 is a simplified illustration of an expander element being pulled back through an aspiration tube.

FIG. 28 is a simplified illustration of the expander element being pulled back through the aspiration tube and plastically deforming it.

FIG. 29 is a simplified illustration of the expander element as it is pulled free of the aspiration system after having expanded the diameter of aspiration tube.

FIG. 30 is a simplified illustration of a vacuum aspiration system having a vacuum pump, a vacuum gauge, a collection chamber and a controllable release valve.

FIG. 31 is a simplified illustration of a collection element having a vacuum valve.

FIG. 32 is a simplified illustration of a pulmonary thrombus lodged in pulmonary arteries.

FIG. 33 is a simplified illustration of an expandable thrombus capture device, in accordance with a non-limiting embodiment of the invention.

FIG. 34 is a simplified illustration of the distal end of the expandable thrombus capture device.

FIG. 35 is a simplified illustration of the expandable thrombus capture device being advanced towards a blockage in a body lumen.

FIG. 36 is a simplified illustration of an outer shaft being retracted and exposing the distal end of a support frame.

FIG. 37 is a simplified illustration of the expandable thrombus capture device with its distal section fully open.

FIG. 38 is a simplified illustration of the distal end of expandable thrombus capture device being advanced over the blockage.

FIG. 39 is a simplified illustration of a membrane being twisted around the blockage so as to firmly capture it.

FIG. 40 is a simplified illustration of a thrombus snagging device, in accordance with a non-limiting embodiment of the invention.

FIG. 41 is a simplified illustration of the thrombus snagging device being advanced in a body lumen.

FIG. 42 is a simplified illustration of an outer shaft being pulled proximally to expose a flexible arm.

FIG. 43 is a simplified illustration of the thrombus snagging device in the fully open position.

FIG. 44 is a simplified illustration of the thrombus snagging device in a body lumen and a flexible arm in the fully extended position.

FIG. 45 is a simplified illustration of the thrombus snagging device as it is rotated around its longitudinal axis.

FIG. 46 is a simplified illustration of a device for capturing obstructions in a body lumen, in accordance with another non-limiting embodiment of the invention.

FIG. 47 is a simplified illustration of the device, in which an outer shaft has been pulled proximally backwards.

FIG. 48 is a simplified illustration of the elements of the device as they get exposed by further withdrawing the outer shaft proximally.

FIG. 49 is a simplified illustration of the various elements of the obstruction capture device, after the outer shaft has been completely pulled back.

FIG. 50 is a close-up of the obstruction capture elements.

FIG. 51 is a simplified illustration of the device as the inner shaft is pulled back axially, causing sleeve elements to compress and deform obstruction capture elements radially.

FIG. 52 is a close-up of the obstruction capture elements with the radial arms in their radially open state.

FIG. 53 is a simplified illustration of the device with its obstruction capture elements and radial arms in the radially open state as it is located next to an obstruction.

FIG. 54 is a simplified illustration of the device as the inner shaft moves distally forward in the axial direction, which removes the compressive force on the obstruction capture elements.

FIG. 55 is a simplified illustration of the obstruction as it is captured by the obstruction capture elements of the device.

FIG. 56 is a simplified illustration of the device as it pulls the obstruction from the lumen.

FIGS. 57 and 58 illustrate other configurations which may be used to “pinch” the obstruction.

### DETAILED DESCRIPTION

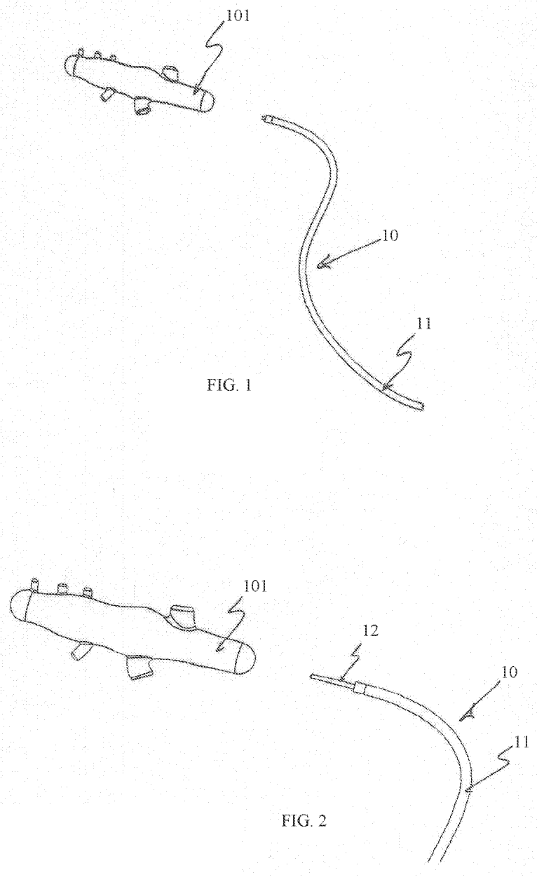

FIG. 1 is a schematic of an obstruction 101 of within a body lumen conduit such as an artery or a vein (conduit not shown for clarification purposes). According to one possible embodiment, a catheter-based treatment device (CBTD) 10 is shown advancing towards an obstruction such as a thrombus. The outer shaft 11 of CBTD 10 is seen.

FIG. 2 shows an inner tube 12 being advanced from within outer shaft 11. Inner tube 12 is advanced towards the obstruction 101.

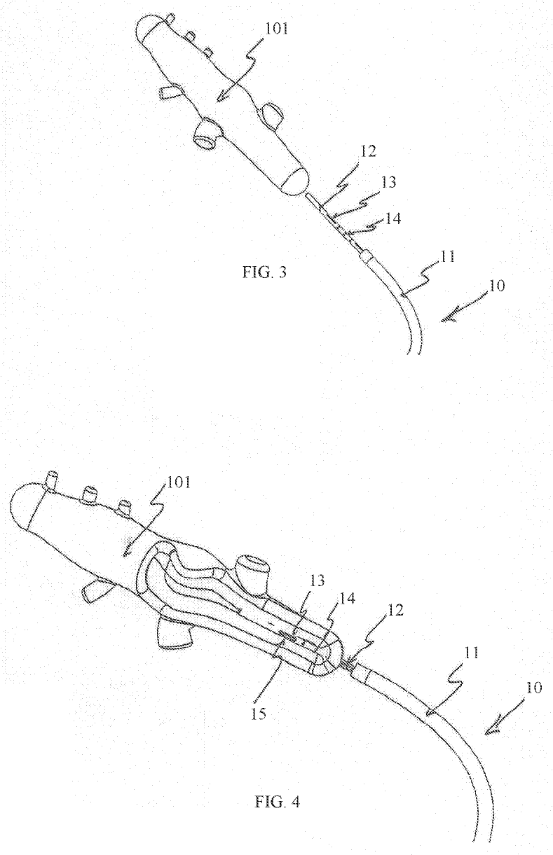

FIG. 3 shows the inner tube 12 having slots 13 and nozzle holes 14. Inner tube 12 can move within outer shaft 11 both in the axial and radial directions.

FIG. 4 is a schematic of inner tube 12 being advanced into obstruction 101 so that both slots 13 and nozzle holes 14 are located within the obstruction.

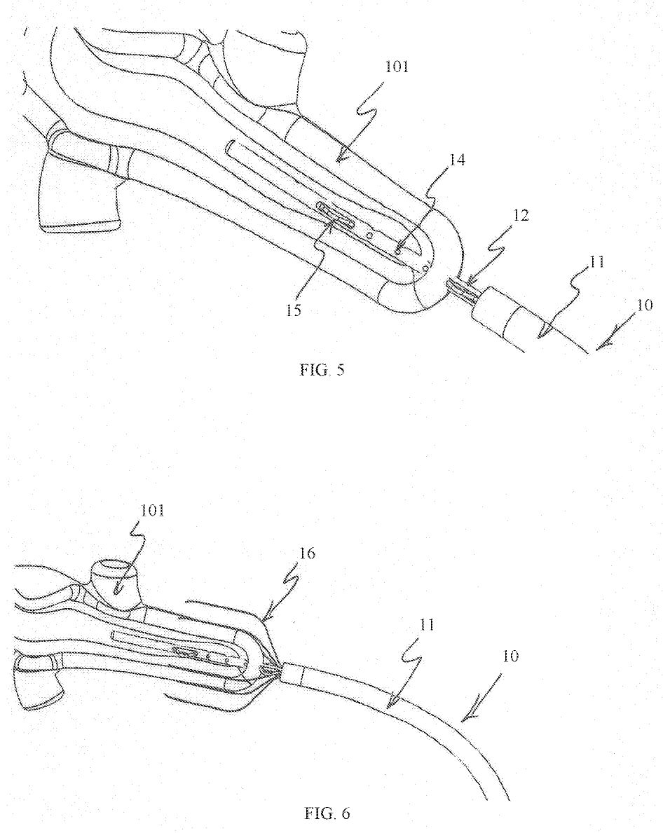

FIG. 5 shows inner tube 12 within obstruction 101. The obstruction has a cutaway section for clarification. Disruption elements 15 are shown extending in a radial direction. These disruption elements can be extended at the operator's discretion to disrupt the body of the disruption 101. In parallel, obstruction melting substances may be applied through nozzle holes 14. The combination of mechanical disruption by the disruption elements 15 and obstruction melting substances applied through nozzle holes 14 may aid in aspirating the obstruction through outer shaft 11.

FIG. 6 shows CBTD 10 with centering element 16 designed to ensure that inner tube 12 is inserted into the obstruction 101 rather than find its way between the obstruction and the body lumen (not shown).

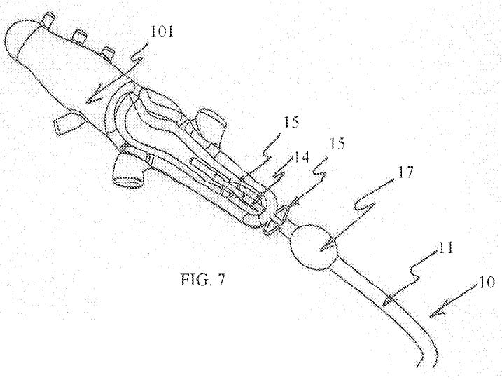

FIG. 7 is a further development of the present embodiment whereby outer shaft 11 has balloon 17 designed to expand and form a seal against the inner diameter of the body lumen, thereby isolating the treatment zone allowing the obstruction melting elements to dwell in the area of the thrombus, thus increasing the effectiveness of the substances.

Accordingly, the present invention provides a catheter based treatment device to treat obstruction in a body lumen having an outer shaft which is connected to an aspiration device at its proximal end. An inner tube is located within the outer shaft, the inner tube having a series of nozzle holes and slots at its distal end. The nozzle holes and slots may be distributed radially at or near the distal end of the inner tube. The slots at the distal end of the inner tube allow disruption elements to extend out in a radial direction into the obstruction so that rotation of the inner tube disrupts the obstruction. An obstruction melting substance such as a lytic material in the case of blood clots may be introduced through the inner tube to help melt the obstruction. The mechanical disruption when combined with the melting material may help in aspirating the obstruction through the outer shaft.

In addition, a pressure profile can be used whereby the negative pressure created by the outer shaft can be combined with the positive pressure of the melting substance as it is introduced through the nozzle holes. Using an oscillating pressure profile when aspirating the obstruction, combined with pulsed application of the melting substance through the nozzle holes may aid in dislodging the obstruction and aspirating it from the lumen without extensive blood loss.

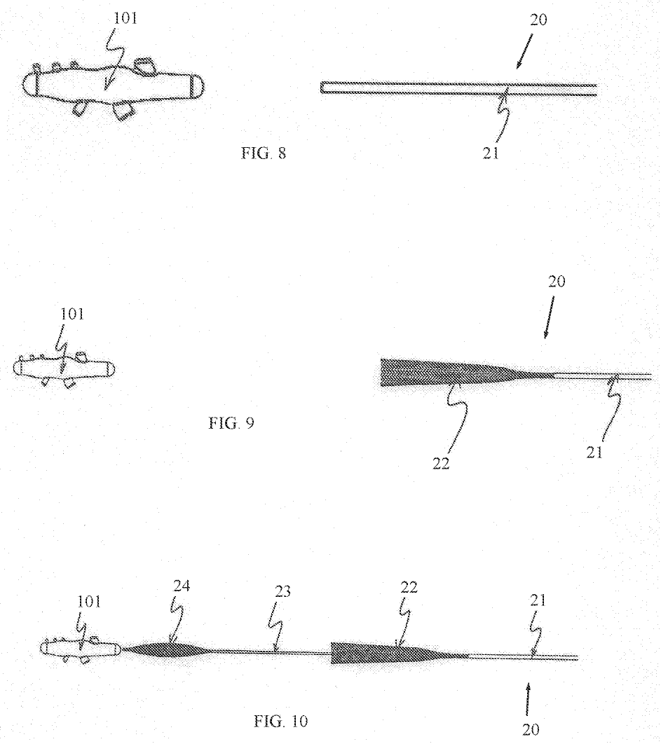

FIG. 8 is another embodiment of the present invention. A CBTD 20 is advanced towards an obstruction 101 in a body lumen (not shown). Outer shaft 21 is shown advancing towards the obstruction.

FIG. 9 is a further depiction of CBTD 20. Outer shaft 21 is pulled caudally, allowing a self-expanding mesh cone 22 to expand and seal against the inner lumen wall.

FIG. 10 shows in inner element extending from within the outer shaft 21 comprising a proximal capture mesh tube 23, capture mesh 24 and distal capture mesh tube 25 which is not shown in this Figure.

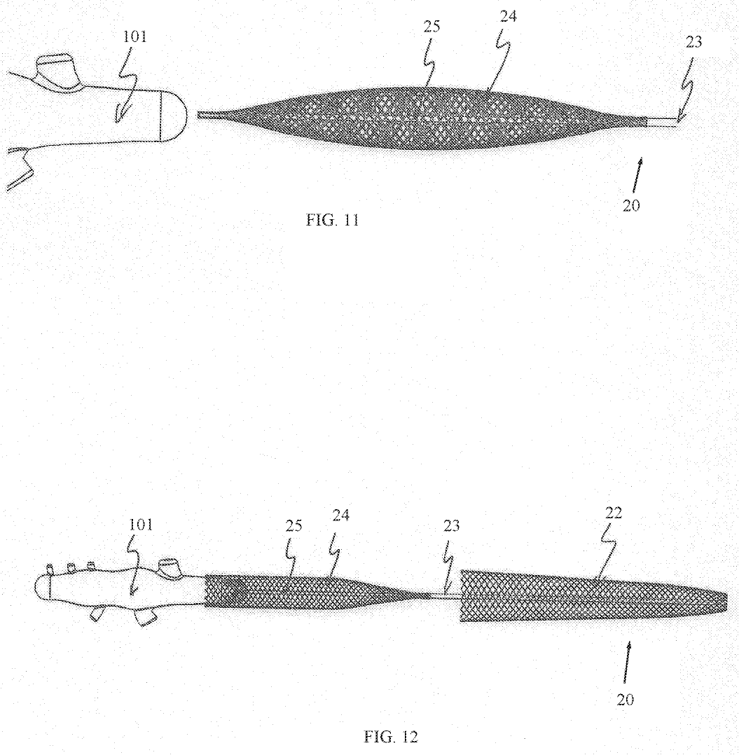

FIG. 11 shows the structure of the distal capture mesh 24. A proximal capture mesh tube 23 is attached to the proximal end of the capture mesh 24 while a distal capture mesh tube 25 is connected to the distal end of the capture mesh 24. Distal capture mesh tube 25 is concentric and is within proximal tube 23 so that distal capture mesh tube 25 can move relative to proximal capture mesh tube 23 both in a radial direction and in an axial direction.

Reference is made to FIG. 12. The distal end of the capture mesh 24 is advanced cranially until it abuts against obstruction 101. At this point, proximal capture mesh tube 23 is advanced cranially while distal capture mesh tube 25 is stationary against the obstruction. This motion causes the capture mesh 24 to fold over itself and engulf part of obstruction 101. At this point, distal capture mesh tube 25 is rotated relative to proximal capture mesh tube 23 causing the capture mesh 24 to tighten radially inward, thereby squeezing the obstruction. The operator can pull the obstruction caudally and into the mesh cone 22. All elements can then be pulled into outer shaft 21. In this embodiment, outer shaft 21 may be connected to an aspiration system at its proximal end to aspirate the obstruction or parts thereof.

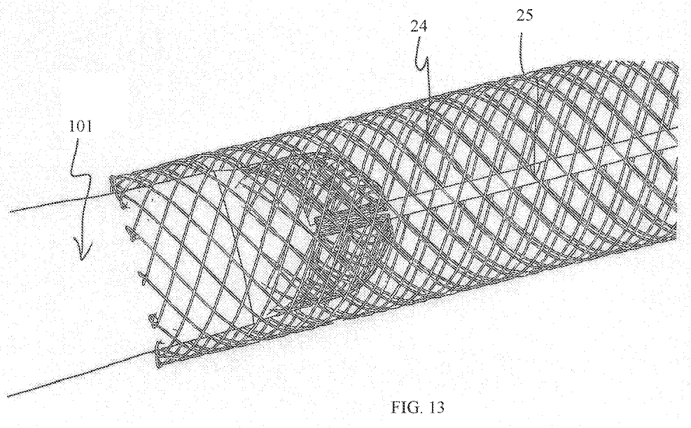

FIG. 13 is a close-up view of the capture mesh 24 as it folded over obstruction 101. The relative motion between distal and proximal capture mesh tubes 25 and 23 respectively causes the tightening of capture mesh 24 around the proximal part of the obstruction.

Accordingly, the present invention provides a catheter based treatment device to treat an obstruction in a body lumen. One embodiment provides an outer shaft, a mesh cone and a distal capture mesh having distal and proximal tubes attached to its respective ends. The capture mesh in advanced until its distal end rests against the obstruction. At this point, the distal capture mesh tube is advanced cranially so that it causes the capture mesh to deform and fold over itself and extend over part of the obstruction. The distal capture mesh tube which is connected to the distal end of the deformed capture mesh is rotated relative to the proximal capture mesh tube causing the capture mesh to tighten around part of the obstruction thus allowing the operator to pull the obstruction free and retract it into the mesh cone and then into the outer shaft. In another specific embodiment of this invention, a vibration element can incorporated into the tube system can be used to aid the relative radial motion of one tube vis-a-vis the other.

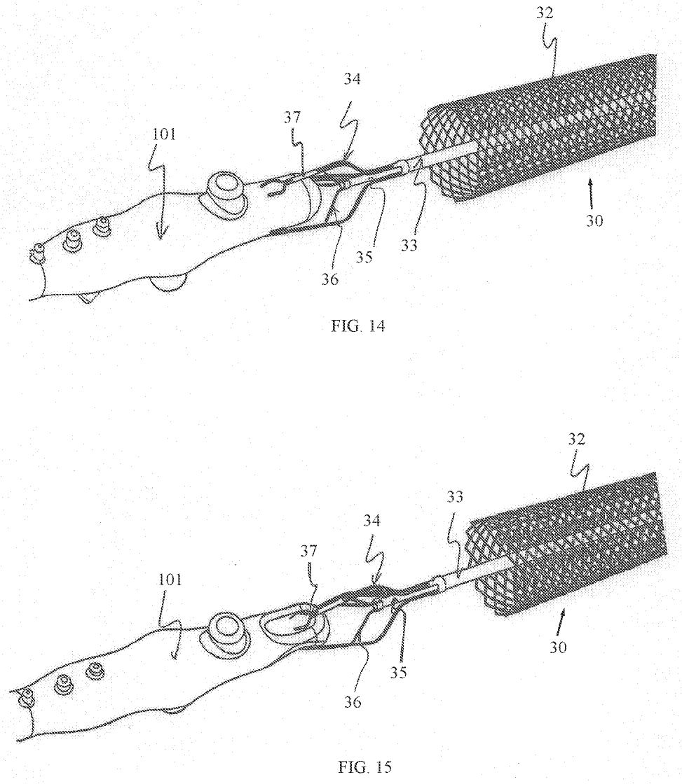

FIG. 14 is an illustration of yet another embodiment of the present invention. A CBTD having a conical cone 32 shown in a deployed state has a grasper element 34 disposed at its distal end. Two concentric tubes 33 and 35 are connected to the grasper 34 so that inner grasping tube 33 is connected to a plurality of operating arms 36. FIG. 14 shows a three-arm configuration. Outer grasping tube 33 is connected to the proximal end of the grasper element. A relative axial motion is affected by pulling inner grasping tube 35 into outer grasping tube 33 thereby causing the operating arms to deform the distal end of the grasper element, closing the grasper arms 37 radially inward grasping obstruction 101 so that it can be pulled into conical mesh 32.

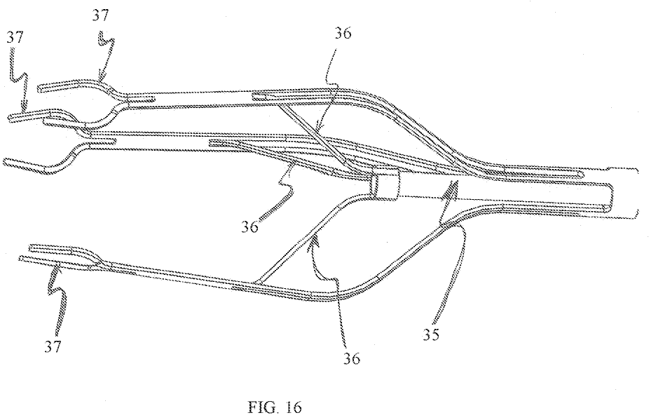

FIG. 15 shows the obstruction 101 grasped in the grasping arms 37.

FIG. 16 is a schematic of a possible configuration of a grasping element having operating arms 36 which are cut from the struts of the grasping arms 37. This configuration shows three grasping arms 37 and operating arms 36 although other configurations are known to a person having ordinary skills in the art. In addition, this embodiment shows operating arms 36 being cut from the struts of grasping arms 37, other configurations exist for this design as well.

Accordingly, the present invention provides a grasping CBTD to grasp and remove an obstruction in a body lumen. The CBTD may be constructed of an outer shaft (not shown), a conical cone to accept the obstruction once it is retracted and a grasping mechanism at its distal end. The grasping mechanism comprises a plurality of grasping elements disposed in a generally radial manner, the grasping elements having proximal and distal ends. The distal ends are used to grasp an obstruction in a body lumen such as a thrombus in a vein or artery. The proximal ends of the grasping elements are connected to a grasping tube. Operating arms are cut from the material of the grasping arms (struts) and are connected to an inner grasping tube which is concentric and internal to the outer grasping tube. The proximal ends of the operating arms are disposed between the proximal and distal ends of the grasper arms such that axial motion between the inner and outer grasping tubes causes the grasper element to collapse inward in a radial direction so as to grasp the obstruction.

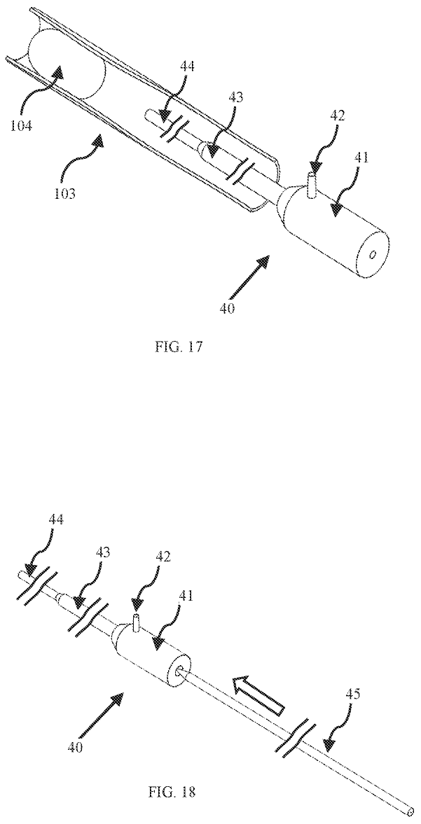

in another aspect of the invention, a deformable aspiration tube is provided for extraction of occlusions from body lumens, as is now described with reference to FIGS. 17-31.

Thrombectomy is a procedure whereby thrombus formations are removed from vasculature by inserting a long tube to the occlusion (thrombus) site and attempting to remove it. In the prior art, the simplest and quickest method of removing the thrombus is by use of an aspiration technique where a negative pressure (vacuum) is generated at the proximal end of the tube which is located outside of the patient's body and where the vacuum generated is sufficient to dislodge the thrombus and suction it into the distal end of the aspiration tube. Navigating the arteries or veins in order to reach the blockage is difficult, especially as the diameter of the aspiration tube increases. On the other hand, aspiration efficacy decreases dramatically as the diameter of the aspiration tube decreases.

In one aspect of the invention, a device and method are provided whereby small diameter aspiration tubes can be inserted and advanced through the vasculature to the target site and then plastically deformed to increase their diameters to the required size. In addition, a vacuum generating system which can predetermine the vacuum level prior to the aspiration action is described.

FIG. 17 shows aspiration system 40 located within a lumen 103 with its distal end just proximal to a thrombus 104. Aspiration system 40 consists of an aspiration hub 41, aspiration port 42 and an aspiration tube having two sections — distal section 44 and proximal section 43 whereby distal section 44 has a smaller diameter than proximal section 43.

FIG. 18 shows an expander element 45 being inserted into aspiration system 40 through aspiration hub 41. Expander element 45 may have a diameter greater or equal to that of proximal section 43 and has a greater diameter than that of distal of distal section 44. Expander element 45 plastically deforms either distal section 44 only, or both distal and proximal sections 44 and 43 depending on its diameter.

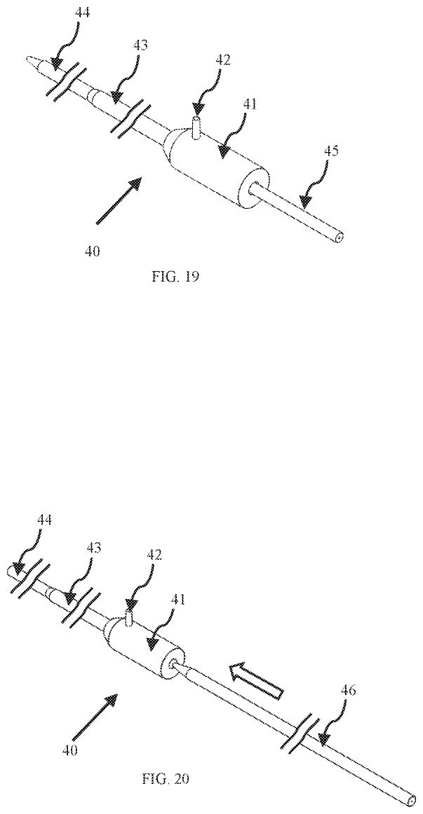

FIG. 19 shows expander element 45 as it is advanced through both distal and proximal sections 44 and 43 until it is proud of the distal end of section 44.

FIG. 20 shows another expander element 46 having a larger diameter of expander element 45 being inserted through aspiration hub 41 to further increase the diameter of either distal section 44 only, or both distal and proximal sections 44 and 43.

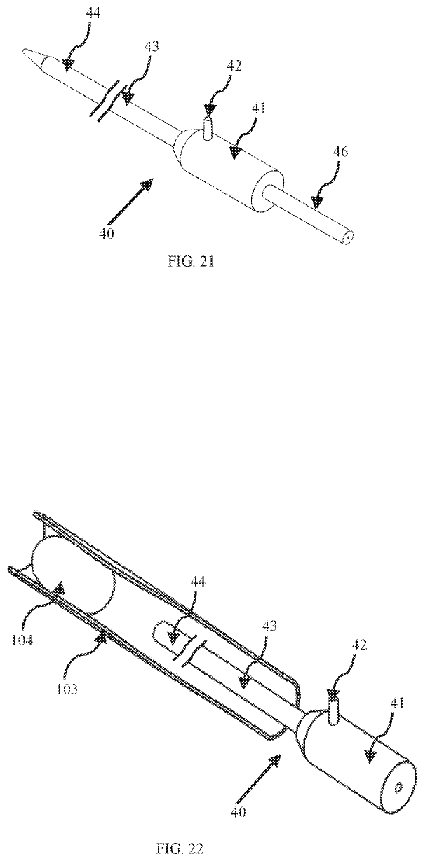

FIG. 21 shows aspiration system 40 with both distal and proximal sections 44 and 43 having approximately the same diameter following the expanding process. The final diameter is determined by the diameter of the expanders used and the characteristics of the materials used to construct the proximal and distal sections 43 and 44. The degree of expansion is controlled by the physician who decides which final size of expander to use.

FIG. 22 shows a fully expanded aspiration system 40 within lumen 103 and just proximal to thrombus 104 prior to aspiration.

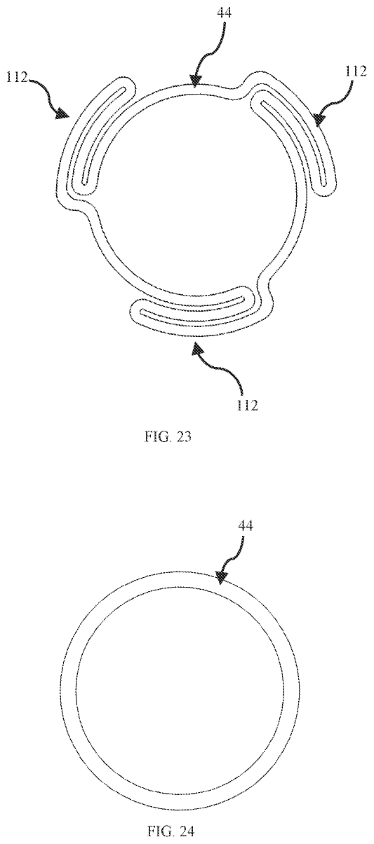

FIG. 23 is a schematic representation of the cross section of distal section 44 whereby folds 112 are used to fold the excess material needed to expand the diameter of distal section 44 from its crimped state to its fully expanded state.

FIG. 24 is a schematic representation of another method of achieving the expansion of distal section 44 whereby the material of distal section 44 is stretched and deformed when expanded. A composite structure may be used in order to manufacture distal section 44 having supportive structures encapsulated or connected to the jacket material such as is known to a person skilled in the art. The composite structure may aid in a uniform plastic deformation of distal section 44.

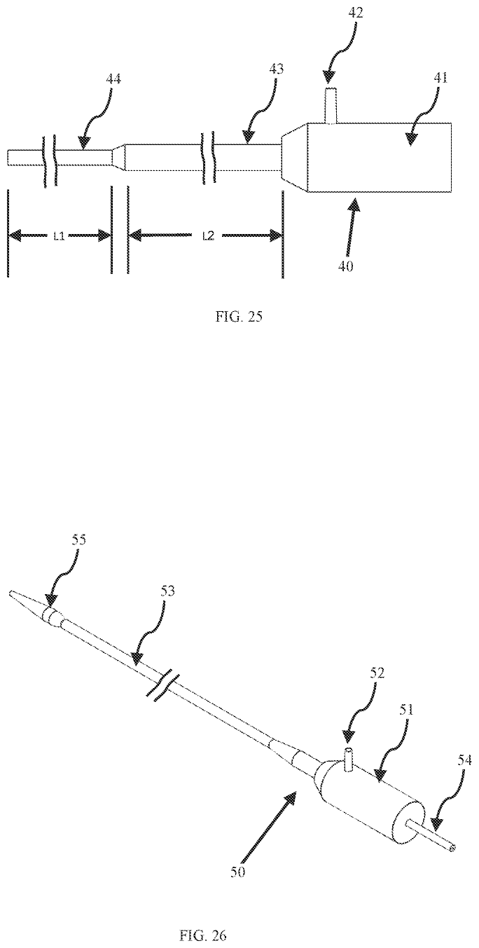

FIG. 25 shows the relations between the lengths of distal section 44 and proximal section 43. L2/L1 can range between 0.1 and 0.8.

FIG. 26 is another embodiment of an expanding aspiration system 50 having an aspiration hub 51 and an aspiration port 52. Aspiration tube 53 has an expander element 54 predisposed within it, the expander element having a diameter generally similar to the internal diameter of aspiration tube 53 such that it can move freely within the aspiration tube. The expander element 54 has a distal tip section 55 whose diameter is greater than that of aspiration tube 53.

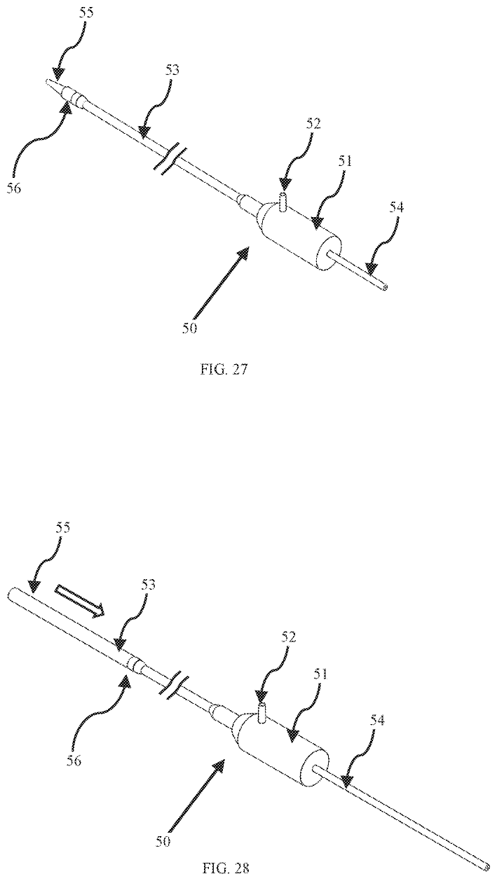

FIG. 27 is a schematic representation of expander element 54 being pulled back through aspiration tube 53 and plastically deforming it causing a bulge 56 which shows the diameter of aspiration tube 53 expanding.

FIG. 28 is a further depiction of expander element 54 being pulled back through aspiration tube 53 and plastically deforming it. Bulge 56 is more proximal now, which means that a longer section of aspiration tube 53 has expanded.

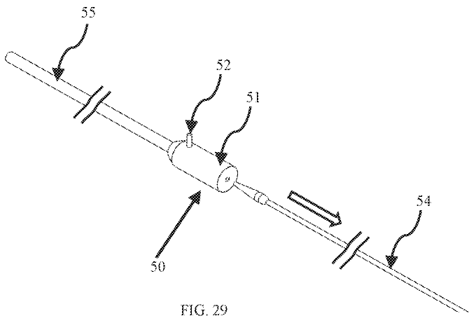

FIG. 29 shows the expander element 54 as it is pulled free of the aspiration system 50 after having expanded the diameter of aspiration tube 53. Aspiration tube may also consist of two or more sections having different diameters in order to facilitate pull-back of expander element 54.

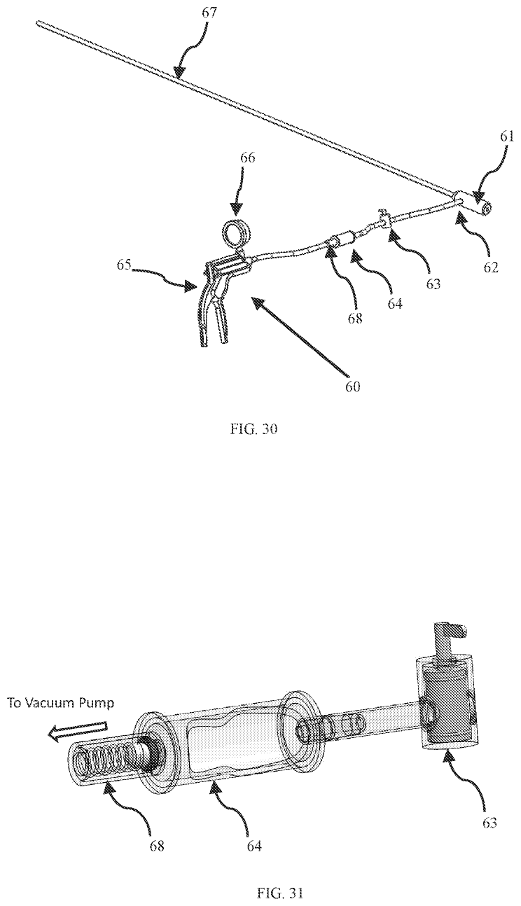

FIG. 30 shows a vacuum aspiration 60 system having a vacuum pump 65, a vacuum gauge 66, a collection chamber 64 and a controllable release valve 63. They are connected in line to an aspiration port 62 which is part of aspiration hub 61. Aspiration tube 67 is connected to aspiration hub 61. The vacuum pump may be actuated until a desired value is reached. Collection chamber 64 has a vacuum valve 68 which allows the pressure within it to increase as desired until the controllable release valve 63 is released. All mater aspirated through the distal end of aspiration tube 67 will be siphoned through and out aspiration port 62 and into collection chamber 64.

FIG. 31 shows collection element 64 having a vacuum valve 68 which is connected to vacuum pump (not shown). The pump can be actuated until the desired vacuum level is reached. The collection chamber 64 is evacuated and remains under vacuum until the controllable release valve 63 is released. At this point, any material located in the system, from aspiration tube 67 backward will be pulled into collection chamber 64. In FIG. 31, vacuum valve 68 is depicted as a spring loaded ball which allows air to be evacuated in one direction only, but alternatively many types of vacuum valves may be used.

In another aspect of the invention, a device for trapping an obstructive mass is provided.

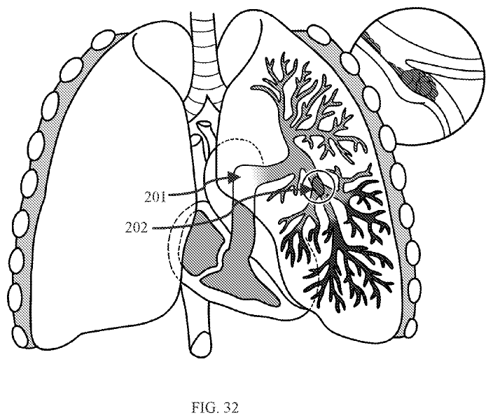

Reference is now made to FIG. 32, which is a schematic representation of a pulmonary thrombus 202 lodged in the pulmonary arteries 201.

FIG. 33 shows an expandable thrombus capture device 70 designed to be introduced in a radially crimped state to the site of a blockage in a body lumen. Device 70 consists of three concentric shafts; An outer shaft 71 into which all elements of device 70 are crimped, a medial shaft 72 and an inner shaft 74. In addition to shafts 71, 72 and 74, device 70 has a radially compressible distal end designed to capture a blockage in a body lumen such as a pulmonary artery. The distal end of the capture device 70 has an open diameter which may be larger than its proximal end essentially forming an open cone. The distal end of device 70 consists of a support frame 79 having an open distal end and is constructed of an elastic material, such as but not limited to, nitinol. The end of the support 79 is constructed to allow the support frame to be radially compressed. Support frame 79 is connected at its proximal end to connecting collar 73 which is turn connected to medial shaft 72. In one embodiment, support frame 79 is connected to connecting collar 73 using a single strut 75 although other connection methods are known to people proficient in the field. The distal end of frame 79 is connected to a flexible coating or membrane 81 which generally forms a cone. The proximal end of the cone is connected to inner shaft 74 such that the proximal and distal ends of the cone are connected to two different concentric shafts which can move relative to one another. As an example, inner shaft 74 can move axially and radially relative to medial shaft 72. The polymer cover may encapsulate barbed elements 80 which are radially disposed and protrude inward facing the axis of the cone. These barbs may also be angled backwards towards the proximal end of the cone so that when the cone is advanced over a blockage in the lumen, the barbs can readily move across the blockage but will be imbed themselves into the blockage when device 70 is retracted. In addition, by inner shaft 74 is moved radially within medial shaft 72, cone 81 is twisted around its central axis, also causing barbs 80 to imbed into the blockage.

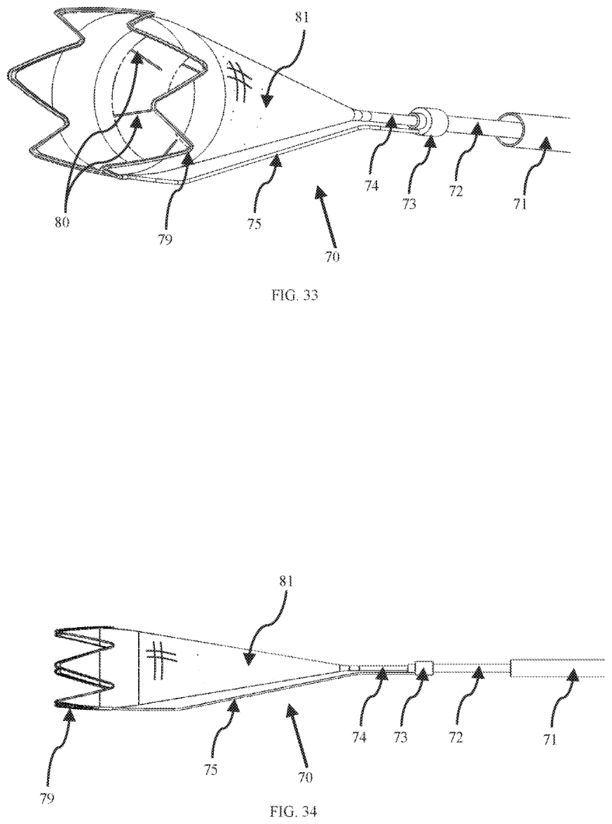

FIG. 34 is another view of the distal end of expandable thrombus capture device 70 showing a side view of flexible membrane 81 connected to support frame 79 on its distal end and to inner shaft 74 in its proximal end. Also shown is strut 75 connecting support frame 79 to connecting collar 73 which is in turn connected to medial shaft 72.

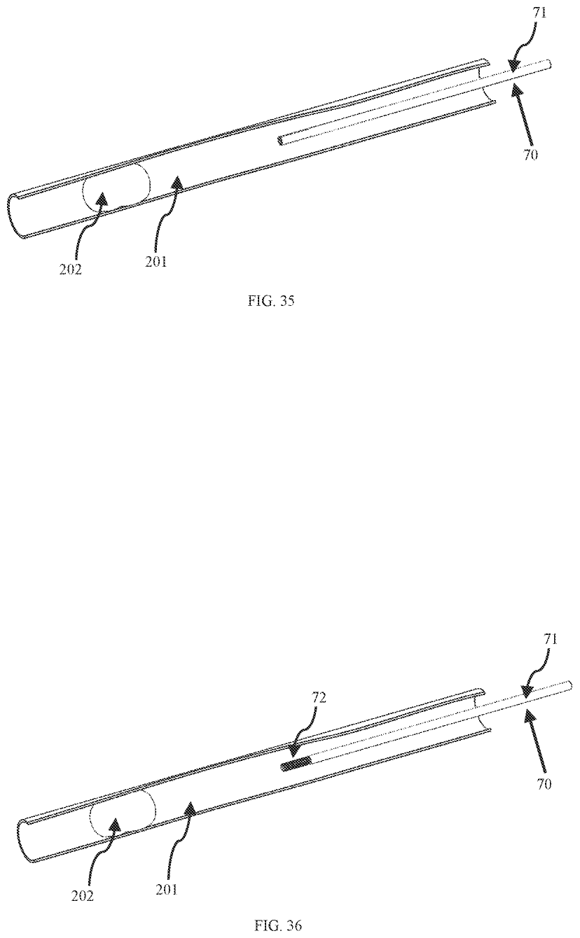

FIGS. 35 to 39 show the steps involved in using the expandable thrombus capture device 70 to remove a blockage in a body lumen.

FIG. 35 shows expandable thrombus capture device 70 being advanced towards a blockage 202 in a body lumen 201.

FIG. 36 shows outer shaft 71 being retracted and exposing the distal end of support frame 79.

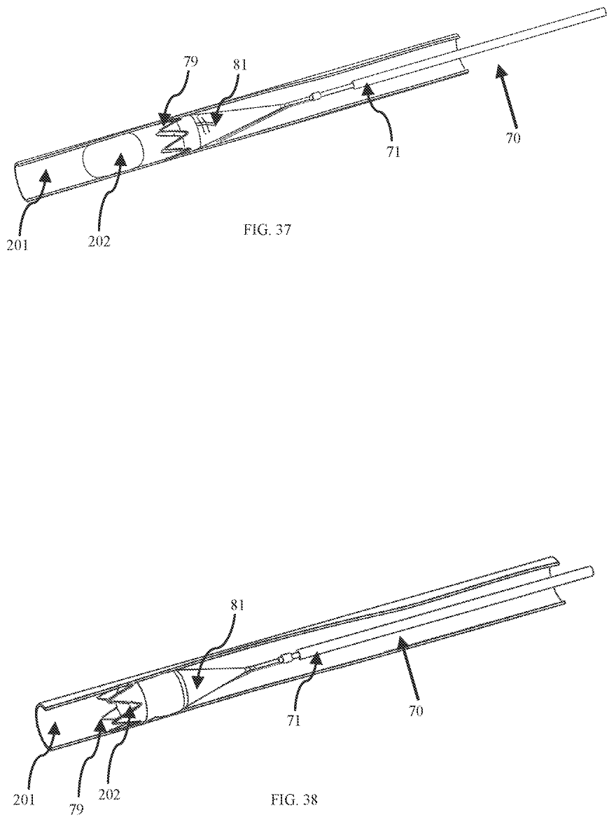

FIG. 37 shows expandable thrombus capture device 70 with its distal section fully open.

FIG. 38 shows the distal end of expandable thrombus capture device 70 being advanced over blockage 202 such that the distal end of support frame 79 extends over the proximal end of blockage 202. At this point, the radial barbs 80 (not shown) may imbed themselves into blockage 202.

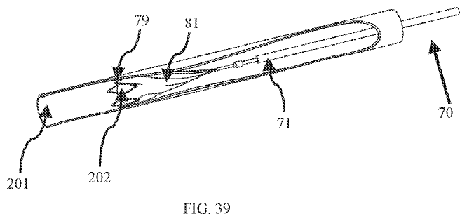

FIG. 39 shows membrane 81 being twisted around blockage 202 so as to firmly capture it. Once blockage 202 is captured, device 70 may be retracted and blockage 202 removed from the body lumen 201.

FIG. 40 is a schematic representation of a thrombus snagging device 90. In this embodiment, a device consisting of an outer shaft 92, an inner shaft 93 connected to a tip 91 is shown. A radially extendable flexible arm 94 is connected to inner shaft 93. The flexible arm 94 can be radially compressed so that outer shaft 92 can encompass all elements of device 90 save tip 91. FIG. 40 depicts device 90 with extendable arm 94 in the radially open position.

FIGS. 41 through 45 depict the steps taken in order to dislodge a thrombus from a body lumen wall.

FIG. 41 shows device 90 being advanced in a body lumen 201. A large thrombus formation 202 is adhered to the lumen wall.

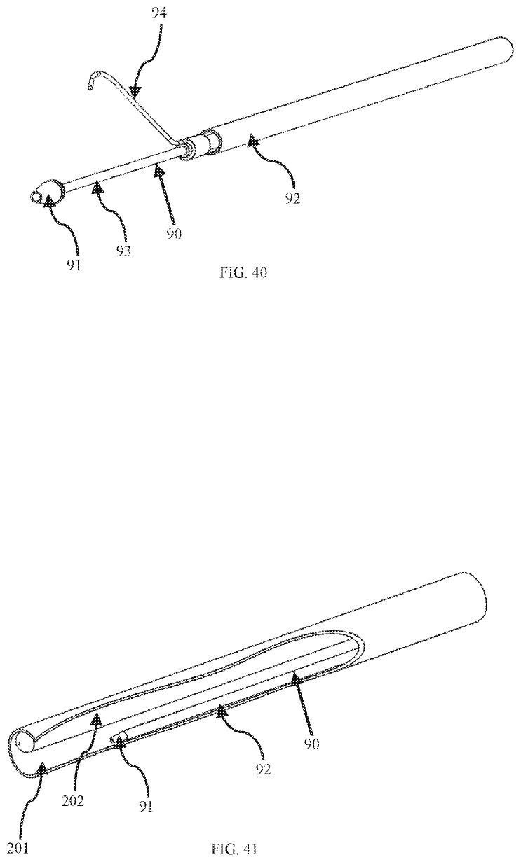

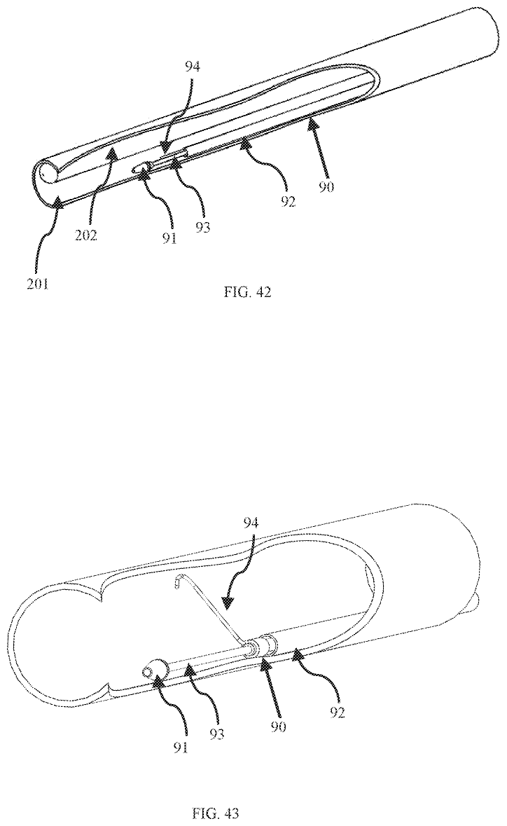

FIG. 42 depicts outer shaft 92 being pulled proximally starting to expose flexible arm 94.

FIG. 43 shows device 90 in the fully open position such that outer shaft 92 is fully retracted and flexible arm 94 is in the fully radial open position. The device is located within the body lumen 201 but thrombus 202 is not shown for clarity purposes.

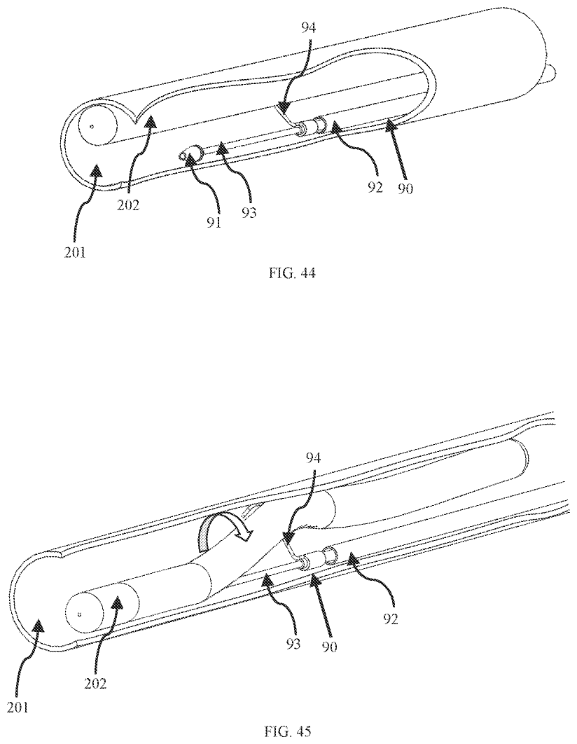

FIG. 44 shows device 90 with in body lumen 201 having flexible arm 94 in the fully extended position. Flexible arm 94 engages the side of thrombus 202.

FIG. 45 shows device 90 as it is rotated around its longitudinal axis. Flexible arm 94 snags thrombus 202 and removes it from the wall of lumen 201. At this point, outer shaft 92 may be advanced, causing flexible arm 94 to grab thrombus 202 allowing the operator to remove the thrombus 202 by retracting the entire device 90. The advancement of outer shaft 92 and the retraction of device 90 are not shown.

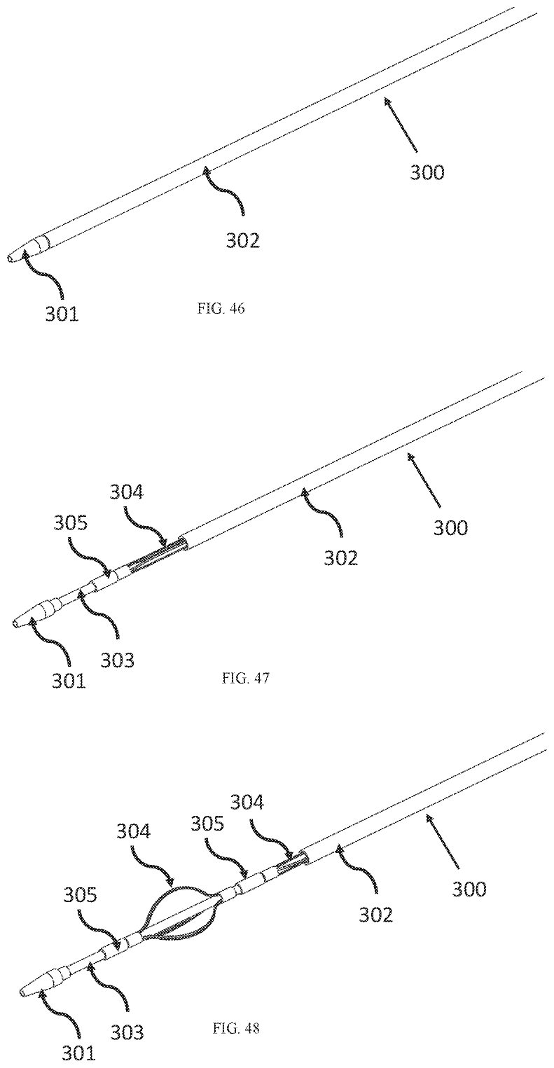

Reference is now made to FIGS. 46-58 which illustrate another obstruction capture device, in accordance with a non-limiting embodiment of the invention.

FIG. 46 illustrates a device 300 designed for capturing obstructions in a body lumen. Device 300 is shown in its radially compressed state within an outer shaft 302 and having a tip 301.

FIG. 47 illustrates device 300, in which outer shaft 302 has been pulled proximally backwards, which partially exposes grasping element 304.

FIG. 48 illustrates the elements of device 300 as they get exposed by further withdrawing outer shaft 302 proximally.

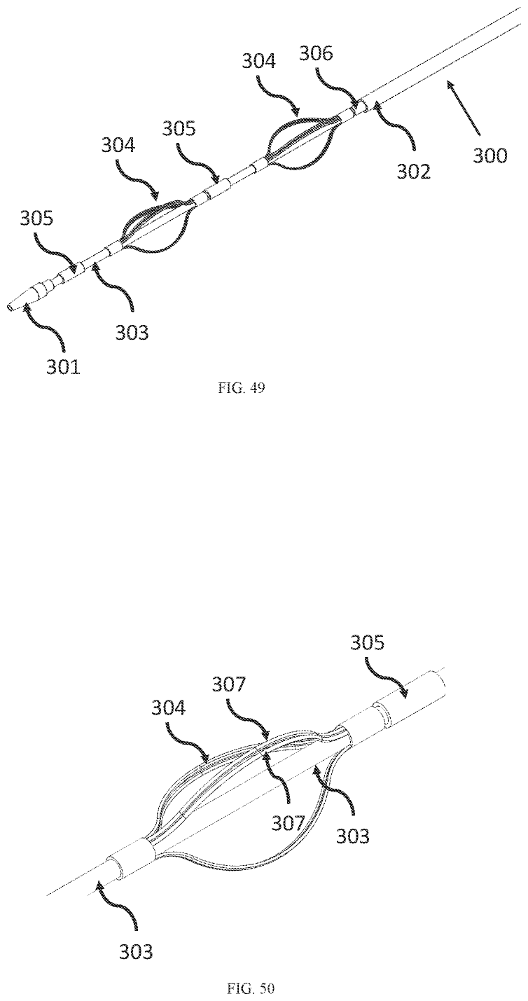

FIG. 49 shows the various elements of obstruction capture device 300, after outer shaft 302 has been completely pulled back. The device may include three concentric shafts: an inner shaft 303, a medial shaft 306 and an outer shaft 302. Inner shaft 303 can move relative to medial shaft 306 which is stationary. Sleeve elements 305 are rigidly connected to inner shaft 303 and have similar diameter dimensions to medial shaft 306 such that they cannot move over or into the medial shaft 306. Obstruction capture elements 304 are located over inner shaft 303 and they can move freely with respect to inner shaft 303 in an axial direction. Once inner shaft 303 is moved in an axial direction against medial shaft 306, sleeve elements 305 compress obstruction capture elements 304 so that they deform (e.g., buckle outward) radially. In this configuration, two obstruction capture elements 304 are shown, but in some configurations, there may be only one obstruction capture element 304, or a plurality of them. The one or more obstruction capture elements 304 form an elastic, radially compressible support frame.

FIG. 50 is a close-up of obstruction capture elements 304. Each capture element 304 may be constructed of a plurality of radially dispersed arm pairs 307 (without limitation, three pairs shown in the illustrated embodiment) which are designed so that each pair is set in a position so that the radial arms 307 of each pair are touching or nearly touching one another in their radially undeformed state.

FIG. 51 shows device 300 as the inner shaft 303 is pulled back axially, causing sleeve elements 305 to compress and deform obstruction capture elements 304 radially, thereby causing radial arms 307 to move apart from each other.

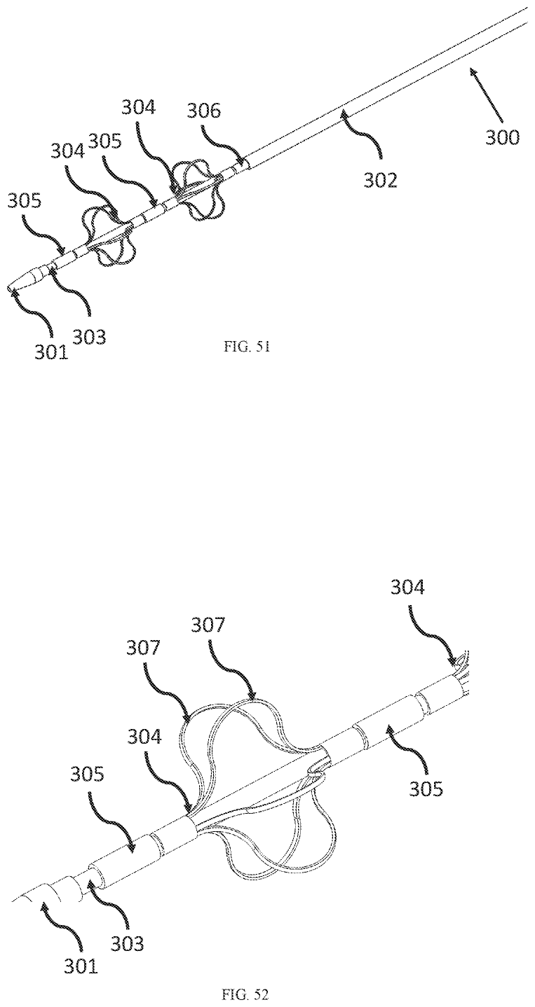

FIG. 52 is a close-up of obstruction capture elements 304 with the radial arms 307 in their radially open state.

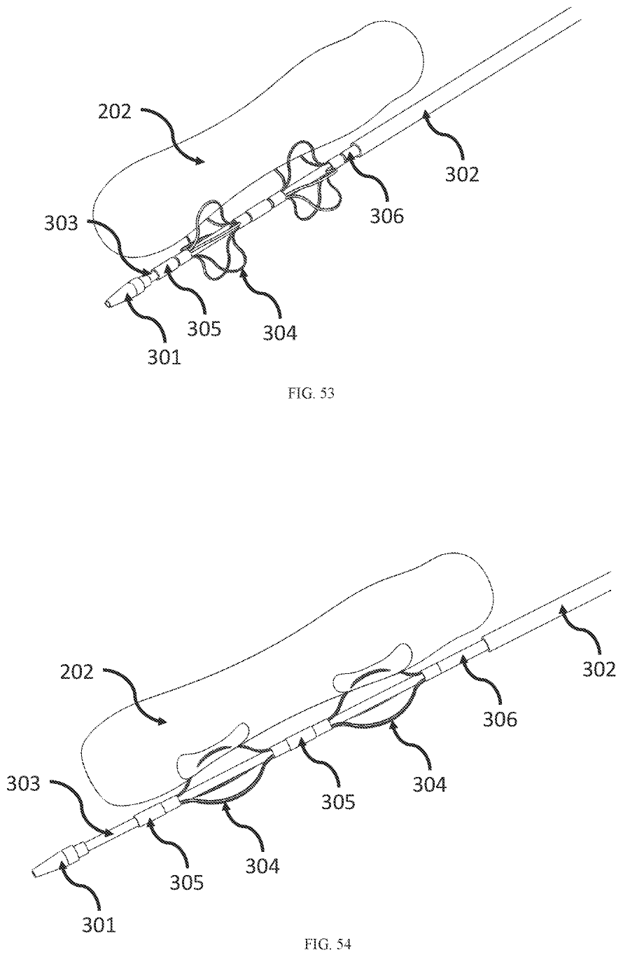

FIG. 53 illustrates device 300 with its obstruction capture elements 304 and radial arms in the radially open state as it is located next to an obstruction 202.

FIG. 54 illustrates device 300 as the inner shaft 303 moves distally forward in the axial direction, which removes the compressive force on obstruction capture elements 304 and causes radial arms 307 to return to their uncompressed state where they touch or almost touch one another. This causes radial arms 307 to “pinch” obstruction 202 and capture portions thereof.

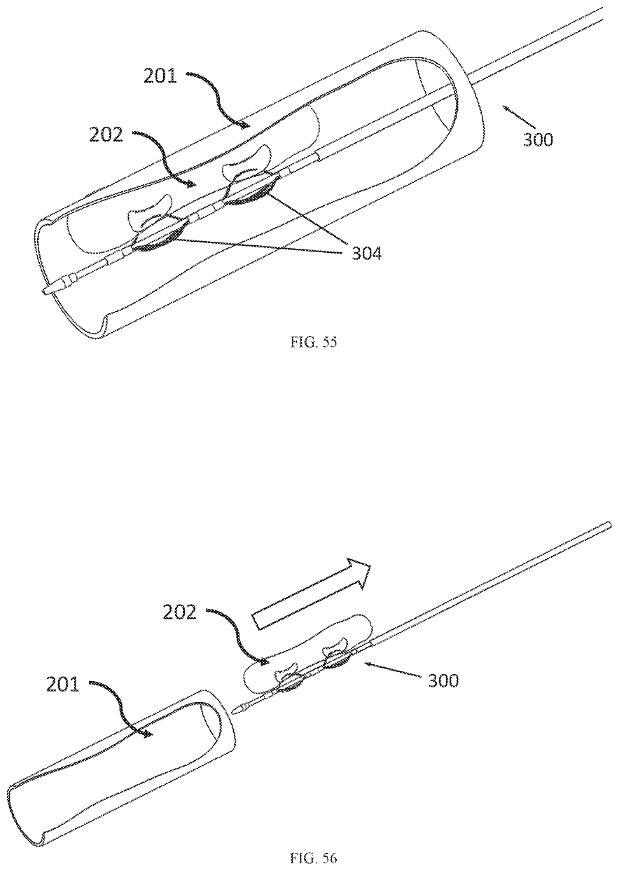

FIG. 55 illustrates the obstruction 202, which had obstructed lumen 201, as it is captured by obstruction capture elements 304 of device 300.

FIG. 56 illustrates device 300 as it is pulled backwards and pulls obstruction 202 from lumen 201.

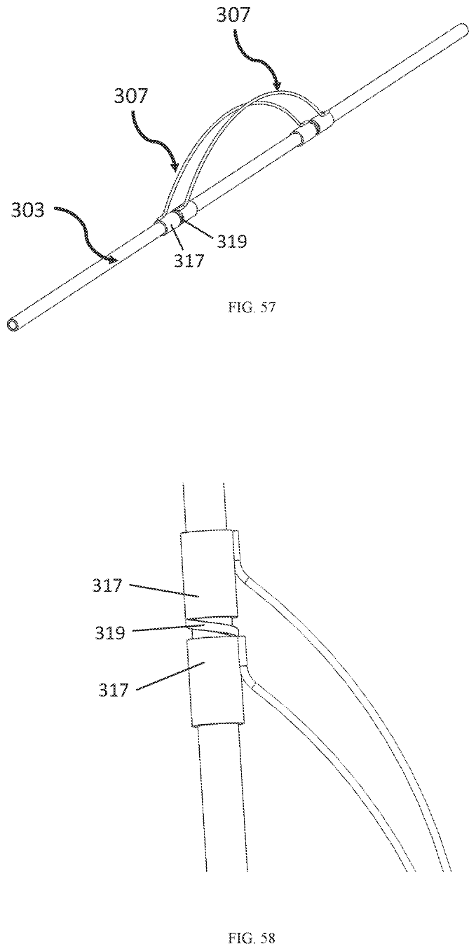

FIGS. 57 and 58 illustrate other configurations which may be used to achieve the same “pinching” action seen in the previous figures. In this case, radial arms 307 are attached to separate collars 317 which are connected via a torsional spring 319. In this configuration, only one pair of radial arms 307 is shown.

1. A device comprising:
an inner tube linearly and rotationally movable in an outer shaft, said inner tube being formed with nozzle holes and slots;
disruption elements extendable outwards through said slots; and
an aspiration device coupled to a proximal portion of said outer shaft.

2. The device according to claim 1, further comprising a lytic agent introduced through said nozzle holes.

3. The device according to claim 1, further comprising an expandable balloon coupled to said outer shaft.

4. The device according to claim 1, wherein said aspiration device comprises an oscillating pressure aspiration device.

5. The device according to claim 1, further comprising a mesh assembly coupled to said outer shaft.

6. The device according to claim 5, wherein said mesh assembly comprises a self-expanding mesh cone.

7. The device according to claim 5, wherein said mesh assembly comprises a proximal capture mesh tube, a capture mesh and a distal capture mesh tube.

8. The device according to claim 1, further comprising a cone and a grasping mechanism coupled to said outer shaft, said grasping mechanism comprising a plurality of outwardly and inwardly movable grasping elements.

9. The device according to claim 1,
wherein said aspiration device comprises an aspiration hub, an aspiration port and an aspiration tube; and
an expander element inserted into said aspiration device through said aspiration hub, said expander element having a size that plastically deforms and enlarges a cross-section of a portion of said aspiration device.

10-13. (canceled)
